(19) United States
(12) Plant Patent Application Publication
(10) Pub. No.: US 2020/0245517 P1
(43) Pub. Date: Jul. 30, 2020

(54) Lagerstroemia plant named 'Chai Berry'

(50) ;

(71) Applicant: Hans A. Hansen, Zeeland, MI (US)

(72) Inventor: Hans A. Hansen, Zeeland, MI (US)

(73) Assignee: Walters Gardens Inc., Zeeland, MI (US)

(21) Appl. No.: 16/350,860

(22) Filed: Jan. 24, 2019

(51) Int. Cl. A01H 6/00

(52) U.S. Cl. PLT/252

(57) ABSTRACT

The new and distinct crape myrtle plant named Lagerstroemia ‘Chai Berry’ has a dense, short, compact, fairly upright habit, is ground hardy to at least USDA zone 6, has glossy, deep medium-green foliage with faint marginal wine blush that emerge cinnamon-colored. The flowers arise from shiny, reddish buds covering the shrub with their rose pink color. The new plant resists leaf spot and powdery mildew and is useful in the landscape as a specimen, en masse, or as a container plant.

Botanical classification: Lagerstroemia (L.) hybrid.

Variety denomination: ‘Chai Berry’.

### STATEMENT REGARDING PRIOR DISCLOSURES UNDER 37 CFR 1.77(b)(6)

The first public disclosure of the claimed plant, in the form of a photograph and brief description on a website operated by Walters Gardens, Inc. on Feb. 1, 2018. After that, on Mar. 12, 2018 the claimed plant was sold by Walters Gardens, Inc., who obtained the plant and all information relating thereto, from the inventor. No plants of Lagerstroemia ‘Chai Berry’ have been sold in this country or anywhere in the world, nor has any disclosure of the new plant been made, more than one year prior to the filing date of this application, and such sale or disclosure within one year was either derived directly or indirectly from the inventor.

### BACKGROUND OF THE INVENTION

The present invention relates to a new and distinct cultivar of Lagerstroemia plant, commonly known as Crape Myrtle, and hereinafter referred to by the cultivar name ‘Chai Berry’ or the “new plant.” ‘Chai Berry’ is grown primarily as an ornamental for landscape use and for use as a potted plant and is the result of an ongoing breeding program to produce new and improved garden worthy plants for the ornamental market. The new plant was the result of open-pollinated seed collected by the inventor in the fall of 2010 in a cultivated landscape in Raleigh, N.C., USA using ‘Whit VI’ U.S. Plant Pat. No. 14,438 (sold under the trademark BURGUNDY COTTON®) as the seed or female parent. The male or pollen parent is unknown, but may have been any one of a number of hybrids or cultivars in the breeding area. The new plant was given the breeder code H10-02-25.

Lagerstroemia ‘Chai Berry’ was initially asexually propagated by stem cuttings at a wholesale perennial nursery in Zeeland, Mich., USA in 2012. The resultant plants from successive generations have demonstrated that the new plant has remained stable and true to type in multiple and successive generations of asexual propagation.

### SUMMARY OF THE INVENTION

Plants of the new cultivar ‘Chai Berry’ have not been observed under all possible environmental conditions. The phenotype may vary somewhat with changes in light, temperature, soil and available moisture and fertility without, however, any variance in genotype.

The following traits have been repeatedly observed and are determined to be unique characteristics of ‘Chai Berry’. Among the characteristics in combination which distinguish ‘Chai Berry’ as a new and distinct cultivar, unique from all other cultivars known to the inventor are:

1. Dense, compact, fairly upright growth habit;

2. Wide glossy foliage emerges cinnamon and matures to medium-green with faint marginal wine blush;

3. Heavily-branched reddish-cinnamon colored stems;

4. Rose-pink flowers in dense panicles open from reddish buds on new growth to cover the shrub;

5. Resistance to Cercospora leaf spot and Erisphe powdery mildew;

6. Ground hardy to at least USDA hardiness zone 6.

The most similar cultivars known to the inventor include: ‘Spiced Plum’ U.S. Plant Pat. No. 29,478, ‘Cool Beans’ U.S. Plant Pat. 29,940, ‘Sweet Macchiato’ U.S. Plant Pat. No. 28,759, and the three copending cultivars ‘Brew Ha Ha’, ‘Dark Roast’ and ‘Lava Java’. ‘Spiced Plum’ is shorter and broader in habit and blooms about a week earlier with flowers that are more raspberry purple. ‘Cool Beans’ has slightly shorter habit and the flower color is a different pink hue. ‘Sweet Macchiato’ is slightly shorter and broader in habit and the flower color is more has lavender pink coloration. ‘Brew Ha Ha’ is slightly shorter in habit and the flower color is a different hue of bubblegum pink. ‘Dark Roast’ is shorter in habit, the leaves are deeper reddish purple and the flowers are a different hue of bright fuchsia pink. ‘Lava Java’ is slightly shorter in habit and the flowers are a deeper reddish hue. The female parent ‘Whit VI’ is much taller, the buds are deeper reddish and the flowers open to white. Comparison with the male parent is not possible since the male parent is unknown.

### BRIEF DESCRIPTION OF THE DRAWINGS

The accompanying color photographs illustrate the flower and foliage characteristics and the overall appearance of a seven-year-old plant of ‘Chai Berry’, growing in a full-sun trial garden in Zeeland, Mich., showing the colors as true as it is reasonably possible to obtain in color reproductions of this type. Colors in the photographs may differ slightly from the color values cited in the detailed botanical description which accurately describe the colors of the new Lagerstroemia.

FIG. 1 shows a plant in a full-sun trial garden in late summer peak flowering.

FIG. 2 shows a close-up for the flowers and buds.

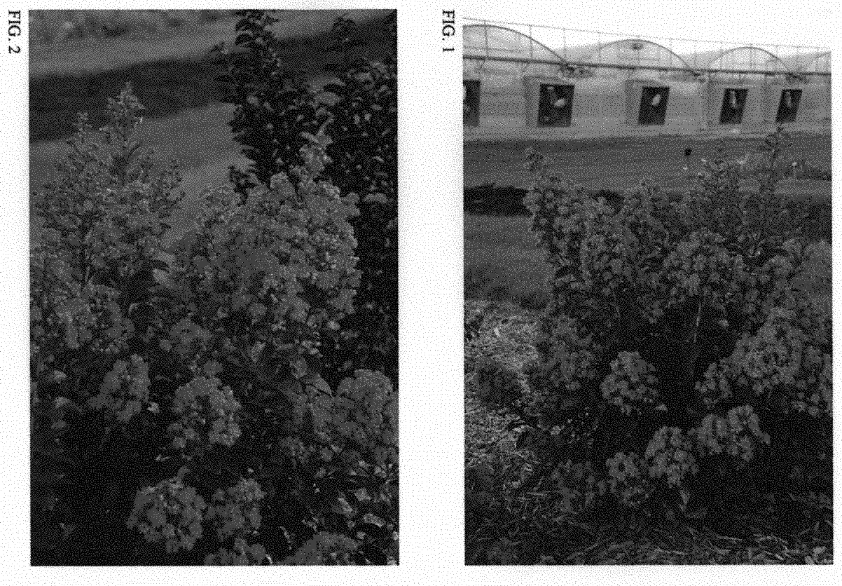

### DETAILED BOTANICAL DESCRIPTION

The following color references are based on the 2015 edition of The Royal Horticultural Society Colour Chart except where common dictionary terms are used. The following observations and size descriptions are of an approximately seven-year-old plant grown in a loamy-sand, full-sun, open trial bed in Zeeland, Mich., USA with supplemental water and fertilizer as needed. The phenotype may vary slightly with different environmental conditions, such as temperature, light, fertility, moisture and maturity levels, but without any change in the genotype.
Botanical classification: Lagerstroemia L.
Parentage: Female, or seed parent ‘Whit VI’; the male, or pollen parent is unknown;
Propagation: Terminal softwood stem cuttings;
Time to initiate roots: About three weeks;
Growth rate: Moderate; finishing from a 65 mm liner in a 3.7 liter container in about 10 to 14 weeks in the summer season;
Plant description: Deciduous, woody, narrow, compact flowering shrub; about six mainly upright to outward primary stems; freely branched;
Root description: Fine, numerous, fibrous, well-branched;
Plant habit: Mostly upright compact mound; about 100.0 cm high from the soil level to the top of the inflorescences; about 110.0 cm wide with no pinching, pruning or plant growth regulators;
Stems: About eleven; to about 70.0 cm long and about 8.0 mm diameter at base; young stems cylindrical with four longitudinal carinae, along line on either side of petioles; basal stems cylindrical with slightly exfoliating bark; highly branched with about 15 to 21 alternate branches held at about 55 to 60 degree angle above horizontal; branches to about 55.0 cm long and 4.0 nun diameter at base;
Stem color: Woody basal portion variable with over wintered stems between RHS 197C and RHS 199D, basal new season stems striated with nearest RHS 174A and RHS 165A; young developing stems striated with nearest RHS 187A, RHS 176D and nearest RHS 175A;
Node: About 35 to 45 per main stem; internode length average about 1.5 cm in main stems.
Node color: Same as surrounding stem;
Foliage description: Sub-opposite to alternate; simple; ovate; margin ciliolate, flat; acute apex; aequilateral, rounded to attenuate base; adaxial and abaxial glabrous and lustrous; to about 6.8 cm long and 3.8 cm wide, average about 5.5 cm long and 3.4 cm wide;
Abaxial leaf color: Young emerging adaxial nearest RHS 146D with strong blushing of nearest RHS 176C, abaxial blend between RHS 176C, RHS 183D and RHS 146D; mature adaxial nearest RHS 139A with occasional marginal blushing nearest RHS 176A, abaxial nearest RHS 147B;
Veins: Pinnate, micro-puberulent adaxial and abaxial;
Vein color: Young emerging adaxial nearest RHS 187B, abaxial nearest RHS 185A with midrib base lighter than RHS 146D and blush lighter than RHS 146D; mature adaxial nearest RHS 183B, abaxial nearest RHS 160C blushed toward perimeter nearest RHS 187C;
Petiole: Short, typically 2.0 mm long and 1.5 mm wide; color young and mature adaxial between RHS 139A and RHS N187A and abaxial nearest RHS N187A;
Inflorescence: Panicle; terminal branched panicles up to about 300 flowers; average about 250 flowers; up to about 30.0 cm long and about 25.0 cm across; beginning late-summer and continuing for about four weeks;
Buds: Globose to ellipsoidal with rounded to slightly apiculate apex and rounded base; lustrous; glabrous; about 7.0 mm long and about 6.5 mm diameter one day prior to opening;
Bud color: Exposed petals nearest RHS 64B; proximal one-half of calyx blend nearest RHS 183C, distal one-half of calyx nearest RHS 181C;
Flowers: Perfect; regular; actinomorphic; terminal panicle; individually about 2.5 cm across and about 19.0 mm long to tip of style, corolla to about 17.0 mm long; lasting about two days;
Flower fragrance: Faintly sweet;
Peduncle: Cylindrical with four longitudinal carinae in proximal portion and cylindrical in distal portion; about 5.0 mm diameter at base below lowest flowering branch, to about 30.0 cm long and 18.0 cm across;
Peduncle color: Variable with position; proximal striated with nearest RHS 174A and RHS 165A; and distal portion striated with nearest RHS 178A and nearest RHS 164B;
Pedicel: Cylindrical; glabrous; lustrous; about 4.5 mm long and 1.0 mm diameter;
Pedicel color: Nearest RHS 187A;
Sepals: Fused in about the basal 3.0 mm; acute apex, entire margin; glabrous and lustrous both adaxial and abaxial; about 7.0 mm long and individually and about 3.0 mm wide at fusion point;
Sepal color: Adaxial basal 3.0 mm nearest RHS 146B, distal portion nearest RHS 181B; abaxial basal nearest RHS 195D, mid-portion nearest RHS 182B and lighter than RHS 192D in the apical 2.0 mm;
Petals: Six; stalked; glabrous; blade ruffled or crisped; margin crisped; blade with rounded apex and auriculate base, to about 9.0 mm across and 9.0 mm long; claw base or stalk adnate to calyx, to about 6.0 mm long and 0.5 mm diameter; overall about 17.0 mm long;
Petal color: Blade adaxial and abaxial nearest RHS 64B; claw nearest between RHS 61C and RHS 61B;
Androecium:
   Stamens.—Typically about 36; six longer and about 30 shorter.
   Filaments.—Shorter stamens to about 9.0 mm long and about 0.2 mm diameter, curved to twisted; longer filaments about 16.0 mm long and about 0.3 mm diameter; color of shorter filaments nearest RHS 61D proximally and distally nearest RHS 155B; color longer filaments between RHS 61C and RHS 61B.
   Anthers.—Dorsifixed; flattened ellipsoid; more developed on longer stamens to about 1.5 mm long and 1.2 mm across, on shorter stamens about 1.0 mm long and about 0.7 mm across; color nearest RHS 13A.
   Pollen.—Abundant on longer stamens; color nearest RHS 14A.
Gynoecium: One; about 19.0 mm long;
   Style.—Cylindrical; glabrous; about 16.0 mm long and 0.5 mm diameter; color nearest RHS 181B.
   Stigma.—Globose; about 0.5 mm diameter; color nearest RHS N186C.
   Ovary.—Superior; globose; lustrous; about 2.0 mm tall and 2.0 mm diameter; color nearest RHS 2D.
Fruit: Globose; dehiscent, loculicidal, penta-valved capsule; about 7.0 mm across and 9.0 mm long; immature color nearest RHS 146B, mature nearest RHS 165A;
Seed: Typically 15 to 36 per fruit; winged; to 6.0 mm long and 3.0 mm across and 1.0 mm thick at embryo; color nearest RHS 165A;
Disease resistance: Lagerstroemia ‘Chai Berry’ has shown resistance to powdery mildew and black leaf spot, Erisphe and Cercospora fungi, respectively. Other resistance beyond that typical for crape myrtle has not been observed. The new plant's root system is capable of withstanding cold temperatures typical of those found in USDA zone 6.

It is claimed:

1. A new and distinct cultivar of crape myrtle plant named Lagerstroemia ‘Chai Berry’ essentially as herein illustrated and described.
